(19) United States
(12) Patent Application Publication
(10) Pub. No.: US 2024/0299270 A1
(43) Pub. Date: Sep. 12, 2024

(54) SKIN CARE COMPOSITIONS

(71) Applicant: Colgate-Palmolive Company, New York, NY (US)

(72) Inventors: Savitha Rajagopal, Chandler, AZ (US); Ewelina Lesniak, North Brunswick, NJ (US); Melissa Moy, Staten Island, NY (US)

(73) Assignee: Colgate-Palmolive Company, New York, NY (US)

(21) Appl. No.: 18/599,930

(22) Filed: Mar. 8, 2024

Related U.S. Application Data
(60) Provisional application No. 63/489,017, filed on Mar. 8, 2023.

(51) Int. Cl. A61K 8/368; A61K 8/34; A61K 8/365; A61K 8/96; A61K 8/9789

(52) U.S. Cl. CPC A61K 8/368; A61K 8/347; A61K 8/365; A61K 8/96; A61K 8/9789

(57) ABSTRACT

Personal care compositions and, for instance, skin care peels are described herein. In accordance with certain aspects, provided is a personal care composition comprising a retinoid, a beta hydroxy acid and/or a salt thereof; and a meroterpene; optionally wherein the personal care composition is substantially free, or free, of resorcinol and/or hydroquinone.

### CROSS-REFERENCE TO RELATED APPLICATIONS

This application claims the benefit of priority from U.S. Provisional Patent Application No. 63/489,017, filed Mar. 8, 2023, and entitled, “Personal Care Compositions,” the content of which is hereby incorporated herein in its entirety, for all purposes.

### BACKGROUND

Skincare has many benefits besides recuperating a youthful and glowing appearance. Skin exfoliates dead skin cells naturally every day, however as a result of the aging process, sun damage or exposure to environmental pollutants, the natural peeling process slows and may stop altogether. Our skin becomes dull, dry, flaky and wrinkled. By gently exfoliating the buildup of dead skin, the firmness, the beauty and the even-toned skin are restored.

Many personal care products currently available to consumers are directed primarily to improving the health and/or appearance of skin. For example, there are a variety of topical skin care products available that are directed to delaying, minimizing, or even eliminating skin dryness, skin wrinkling, and other histological changes commonly associated with the aging of skin or environmental damage to human skin. As a result, the sale of personal care products has become a booming business in youth-conscious societies.

There is a growing need for personal care formulations with anti-ageing exfoliating and brightening effects, which bring out a healthy looking radiance while evening skin tone.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Aspects of the invention are directed to personal care compositions and, in some instances, skin care peels. In accordance with an aspect, provided is a personal care composition comprising oleyl adapalate, a beta hydroxy acid and/or a salt thereof; and a humectant. The personal care composition may, in some embodiments, be in the form of a skin care peel.

In accordance with another aspect, provided is a personal care composition comprising oleyl adapalate, an alpha hydroxy acid and/or salt thereof, and saccharide isomerate.

In accordance with yet a further aspect, provided is a personal care composition comprising oleyl adapalate, an alpha hydroxy acid and/or salt thereof, and lipoic acid and/or a salt thereof.

A list of non-limiting, example embodiments is provided below.

In accordance with embodiment 1, provided is a personal care composition comprising:
oleyl adapalate;
a beta hydroxy acid and/or a salt thereof; and
a humectant.

In accordance with embodiment 2, provided is a personal care composition according to embodiment 1, wherein the personal care composition is a skin care peel.

In accordance with embodiment 3, provided is a personal care composition according to embodiment 1 or embodiment 2, wherein the beta hydroxy acid and/or a salt thereof comprises salicylic acid, beta hydroxybutanoic acid, tropic acid, trethocanic acid, a salt thereof, or a combination of two or more thereof.

In accordance with embodiment 4, provided is a personal care composition according to any foregoing embodiment, wherein the beta hydroxy acid and/or a salt thereof comprises salicylic acid, a salt thereof, or a combination of thereof.

In accordance with embodiment 5, provided is a personal care composition according to any foregoing embodiment, wherein the beta hydroxy acid and/or a salt thereof is present in amount from about 5 to about 25 wt. %, based on the total weight of the personal care composition.

In accordance with embodiment 6, provided is a personal care composition according to any foregoing embodiment further comprising an alpha hydroxy acid and/or salt thereof.

In accordance with embodiment 7, provided is a personal care composition according to embodiment 6, wherein the alpha hydroxy acid and/or salt thereof comprises mandelic acid, lactic acid, malic acid, tartaric acid, phytic acid, a salt thereof, or a combination of two or more thereof.

In accordance with embodiment 8, provided is a personal care composition according to embodiment 6 or embodiment 7, wherein the alpha hydroxy acid and/or salt thereof is present in an amount ranging from about 5 to about 25 wt. %, based on the total weight of the personal care composition.

In accordance with embodiment 9, provided is a personal care composition according to any foregoing embodiment, further comprising an anti-acne agent selected from keratolytics, retinoids, tretinoin, adapalene, tazarotene, lipoic acid, benzoyl peroxide, triclosan, chlorhexidine gluconate, octopirox, tetracycline, 2,4,4′-trichloro-2′-hydroxy diphenyl ether, 3,4,4′-trichlorobanilide, nicotinamide, tea tree oil, rofecoxib, azelaic acid and its derivatives, phenoxyethanol, phenoxypropanol, phenoxisopropanol, ethyl acetate, clindamycin, erythromycin, meclocycline, sebostats, Minocycline, Moxifloxacin, N-acetylcystein, Nadifloxacin, octopirox, Prulifloxacin, Sitafloxacin, sodium sulfacetamide, spirinolactone, sulfacetamide, sulfur, tazarotene, tretinoin, triclosan, ulifloxacin, metronidazole, ornidazole, urea, and a combination of two or more thereof.

In accordance with embodiment 10, provided is a personal care composition according to any foregoing embodiment further comprising an antioxidant selected from amellia japonica extract, quercetin, green tea, soy, milk thistle, algae, aloe, angelica, bitter orange, coffee, goldthread, grapefruit, hoellen, honeysuckle, Job's tears, lithospermum, mulberry, peony, puerarua, rice, safflower, and a combination of two or more thereof.

In accordance with embodiment 11, provided is a personal care composition according to any foregoing embodiment, wherein the humectant is selected from sorbitol, butylene glycol, 1,3-propanediol, glycerin, polyoxyethylene glycol, polyglutamic acid, saccharide isomerate, and a combination of two or more thereof.

In accordance with embodiment 12, provided is a personal care composition according to any foregoing embodiment, wherein the humectant comprises saccharide isomerate.

In accordance with embodiment 13, provided is a personal care composition according to embodiment 11 or embodiment 12, wherein the humectant is present in an amount from about 0.01 to about 5 wt. %, based on the total weight of the personal care composition.

In accordance with embodiment 14, provided is a personal care composition comprising:
oleyl adapalate;
an alpha hydroxy acid and/or salt thereof; and
saccharide isomerate.

In accordance with embodiment 15, provided is a personal care composition according to embodiment 14, wherein the alpha hydroxy acid and/or salt thereof is selected from mandelic acid, lactic acid, malic acid, tartaric acid, phytic acid, a salt thereof, and a combination of two or more thereof.

In accordance with embodiment 16, provided is a personal care composition according to embodiment 14 or embodiment 15 further comprising a beta hydroxy acid and/or salt thereof, the beta hydroxy acid and/or salt thereof comprising salicylic acid, beta hydroxybutanoic acid, tropic acid, trethocanic acid, a salt thereof, or a combination of two or more thereof.

In accordance with embodiment 17, provided is a personal care composition according to any foregoing embodiment further comprising licorice extract.

In accordance with embodiment 18, provided is a personal care composition comprising:
oleyl adapalate;
an alpha hydroxy acid and/or salt thereof; and
lipoic acid and/or a salt thereof.

In accordance with embodiment 19, provided is a personal care composition according to embodiment 18, wherein the alpha hydroxy acid and/or salt thereof is selected from mandelic acid, lactic acid, malic acid, tartaric acid, phytic acid, a salt thereof, and a combination of two or more thereof.

In accordance with embodiment 20, provided is a personal care composition according to embodiment 18 or embodiment 19, further comprising a beta hydroxy acid and/or salt thereof selected from salicylic acid, beta hydroxybutanoic acid, tropic acid, trethocanic acid, a salt thereof, and a combination of two or more thereof.

In accordance with embodiment 21, provided is a personal care composition according to any one of embodiments 18 to 20, further comprising licorice extract.

In accordance with embodiment 22, provided is a personal care composition comprising:
a retinoid;
bakuchiol;
an alpha hydroxy acid, or a salt thereof; and
a cosmetically acceptable carrier.

In accordance with embodiment 23, provided is a personal care composition according to embodiment 22, wherein the alpha hydroxy acid, or salt thereof, is selected from mandelic acid, lactic acid, malic acid, tartaric acid, phytic acid, a salt thereof, and a combination of two or more thereof.

In accordance with embodiment 24, provided is a personal care composition according to embodiment 22 or embodiment 23, wherein the retinoid comprises oleyl adapalate, or a derivative thereof.

In accordance with embodiment 25, provided is a personal care composition according to any one of embodiments 22 to 24, further comprising an anti-acne agent selected from keratolytics, retinoids, tretinoin, adapalene, tazarotene, lipoic acid, benzoyl peroxide, triclosan, chlorhexidine gluconate, octopirox, tetracycline, 2,4,4′-trichloro-2′-hydroxy diphenyl ether, 3,4,4′-trichlorobanilide, nicotinamide, tea tree oil, rofecoxib, azelaic acid and its derivatives, phenoxyethanol, phenoxypropanol, phenoxisopropanol, ethyl acetate, clindamycin, erythromycin, meclocycline, sebostats, Minocycline, Moxifloxacin, N-acetylcystein, Nadifloxacin, octopirox, Prulifloxacin, Sitafloxacin, sodium sulfacetamide, spirinolactone, sulfacetamide, sulfur, tazarotene, tretinoin, triclosan, ulifloxacin, metronidazole, ornidazole, urea, and a combination of two or more thereof.

In accordance with embodiment 26, provided is a personal care composition according to any one of embodiments 22 to 25, further comprising an antioxidant selected from amellia japonica extract, quercetin, green tea, soy, milk thistle, algae, aloe, angelica, bitter orange, coffee, goldthread, grapefruit, hoellen, honeysuckle, Job's tears, lithospermum, mulberry, peony, puerarua, rice, safflower, and a combination of two or more thereof.

In accordance with embodiment 27, provided is a personal care composition according to any one of embodiments 22 to 26, further comprising a humectant selected from sorbitol, butylene glycol, 1,3-propanediol, glycerin, polyoxyethylene glycol, polyglutamic acid, saccharide isomerate, and a combination of two or more thereof.

In accordance with embodiment 28, provided is a personal care composition according to embodiment 27, wherein the humectant comprises saccharide isomerate.

In accordance with embodiment 29, provided is a personal care composition according to any one of embodiments 22 to 28, wherein the personal care composition is in a form selected from: a peel; a serum; a lotion; a cream; a balm; and an ointment.

In accordance with embodiment 30, provided is a personal care composition according to any foregoing embodiment, having a pH of from about 2.5 to about 4.5.

In accordance with embodiment 31, provided is a personal care composition according to embodiment 30, wherein the pH is from about 3.1 to about 4.1.

In accordance with embodiment 32, provided is a method for treating, preventing or ameliorating a symptom associated with: acne; post-acne scars; post-acne texture and tone; hyperpigmentation; clogged pores; and/or inflammation, comprising: applying a personal care composition according to any foregoing embodiment to a skin surface of a subject in need thereof.

In accordance with embodiment 33, provided is a use of any one of the compositions described herein for treating or preventing acne; post-acne scars; post-acne texture and tone; hyperpigmentation; clogged pores; and/or inflammation.

### BRIEF DESCRIPTION OF THE DRAWINGS

FIG. 1 is a bar graph showing the effect of a non-limiting, example skin care composition on TGFB2 and VEGF expression in accordance with aspect of the invention.

FIG. 2 is a bar graph showing the effect of a non-limiting, example skin care composition on AQP3 expression in accordance with aspect of the invention.

FIG. 3 is a bar graph showing the effect of a non-limiting, example skin care composition on sebum, erythema index, texture count, and pore size in accordance with aspect of the invention.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to and can be employed in other apparatuses and methods. Before explaining the disclosed embodiments of the present invention in detail, it is to be understood that the invention is not limited in its application to the details of any particular embodiment disclosed herein. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc.

The term “about” when referring to a number means any number within a range of 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the personal care composition. The symbol “°” refers to a degree, such as a temperature degree or a degree of an angle. The symbols “h”, “min”, “mL”, “nm”, and “μm” refer to hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

When referring to chemical structures, and names, the symbols “C”, “H”, and “O” mean carbon, hydrogen, and oxygen, respectively. The symbols “—”, “═” and “≡” mean single bond, double bond, and triple bond, respectively.

“Volatile”, as used herein, means having a flash point of less than about 100° C. “Non-volatile”, as used herein, means having a flash point of greater than about 100° C.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

The phrases, “a mixture thereof,” “a combination thereof,” or a combination of two or more thereof” do not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.” Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

All components and elements positively set forth in this disclosure can be negatively excluded from the claims. In other words, the personal care compositions of the present disclosure can be free or essentially free of all components and elements positively recited throughout the present disclosure. In some instances, the personal care compositions of the present disclosure may be substantially free of non-incidental amounts of the ingredient(s) or compound(s) described herein. A non-incidental amount of an ingredient or compound is the amount of that ingredient or compound that is added into the personal care composition by itself. For example, a personal care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the personal care composition includes both components (or the composition includes more than two components that overlap), an overlapping compound does not represent more than one component. For example, certain compounds may be characterized as both a beta hydroxy acid and an anti-acne agent. If a particular personal care composition includes salicylic acid, which may be characterized as both a beta hydroxy acid and an anti-acne agent, the salicylic acid will serve only as either a beta hydroxy acid or an anti-acne agent—not both.

For readability purposes, the chemical functional groups are in their adjective form; for each of the adjectives, the word “group” is assumed. For example, the adjective “alkyl” without a noun thereafter, should be read as “an alkyl group.”

Aspects of the invention are directed to personal care compositions and, in some instances, skin care peels. In accordance with an aspect, provided is a personal care composition comprising oleyl adapalate, a beta hydroxy acid and/or a salt thereof; and a humectant. In accordance with another aspect, provided is a personal care composition comprising oleyl adapalate, an alpha hydroxy acid and/or salt thereof, and saccharide isomerate. In accordance with a further aspect, provided is a personal care composition comprising oleyl adapalate, an alpha hydroxy acid and/or salt thereof, and lipoic acid and/or a salt thereof. The personal care composition may, in some embodiments, be a skin care peel.

Suitable components, such as those listed below, may be included or excluded from the formulations for the personal care compositions depending on the specific combination of other components, the form of the personal care compositions, and/or the use of the formulation (e.g., a serum, a skin care peel, etc.). The personal care composition may be a skin care composition, such as a skin care peel.

The personal care compositions may comprise oleyl adapalate in an amount that may vary. Preferably, the personal care composition includes an effective amount of oleyl adapalate. In some instances, the amount of oleyl adapalate present in the personal care composition is from about 0.05 to about 7 wt. %, based on the total weight of the personal are composition. For example, the personal care composition may include oleyl adapalate in an amount from about 0.05 to about 5 wt. %, about 0.05 to about 3 wt. %, about 0.05 to about 2 wt. %, about 0.05 to about 1 wt. %, about 0.05 to about 0.75 wt. %, about 0.05 to about 0.5 wt. %, about 0.05 to about 0.25 wt. %, about 0.05 to about 0.1 wt. %; from about 0.1 to about 7 wt. %, about 0.1 to about 5 wt. %, about 0.1 to about 3 wt. %, about 0.1 to about 2 wt. %, about 0.1 to about 1 wt. %, about 0.1 to about 0.75 wt. %, about 0.1 to about 0.5 wt. %, about 0.1 to about 0.25 wt. %; from about 0.15 to about 7 wt. %, about 0.15 to about 5 wt. %, about 0.15 to about 3 wt. %, about 0.15 to about 2 wt. %, about 0.15 to about 1 wt. %, about 0.15 to about 0.75 wt. %, about 0.15 to about 0.5 wt. %, about 0.15 to about 0.25 wt. %; from about 0.4 to about 7 wt. %, about 0.4 to about 5 wt. %, about 0.4 to about 3 wt. %, about 0.4 to about 2 wt. %, about 0.4 to about 1 wt. %, about 0.4 to about 0.75 wt. %; from about 0.8 to about 7 wt. %, about 0.8 to about 5 wt. %, about 0.8 to about 3 wt. %, about 0.8 to about 2 wt. %, about 0.8 to about 1 wt. %; from about 1.2 to about 7 wt. %, about 1.2 to about 5 wt. %, about 1.2 to about 3 wt. %, about 1.2 to about 2 wt. %; from about 1.6 to about 7 wt. %, about 1.6 to about 5 wt. %, about 1.6 to about 3 wt. %, about 1.6 to about 2 wt. %; from about 2 to about 7 wt. %, about 2 to about 5 wt. %, about 2 to about 3 wt. %, or any range or subrange thereof, based on the total weight of the personal care composition.

The personal care compositions typically comprise one or more beta hydroxy acid(s) and/or salt(s) thereof, e.g., in an amount from about 5 to about 25 wt. %, based on the total weight of the personal care composition. For example, the total amount of beta hydroxy acid(s) and/or salt(s) thereof present in the personal care composition may be from about 5 to about 22 wt. %, about 5 to about 19 wt. %, about 5 to about 17 wt. %, about 5 to about 15 wt. %, about 5 to about 13 wt. %, about 5 to about 11 wt. %, about 5 to about 9 wt. %; from about 8 to about 25 wt. %, about 8 to about 22 wt. %, about 8 to about 19 wt. %, about 8 to about 17 wt. %, about 8 to about 15 wt. %, about 8 to about 13 wt. %, about 8 to about 11 wt. %; from about 10 to about 25 wt. %, about 10 to about 22 wt. %, about 10 to about 19 wt. %, about 10 to about 17 wt. %, about 10 to about 15 wt. %, about 10 to about 13 wt. %; from about 12 to about 25 wt. %, about 12 to about 22 wt. %, about 12 to about 19 wt. %, about 12 to about 17 wt. %, about 12 to about 15 wt. %; from about 14 to about 25 wt. %, about 14 to about 22 wt. %, about 14 to about 19 wt. %, about 14 to about 17 wt. %; from about 16 to about 25 wt. %, about 16 to about 22 wt. %, about 16 to about 19 wt. %; from about 18 to about 25 wt. %, about 18 to about 22 wt. %, or any range or subrange thereof, based on the total weight of the personal care composition.

The personal care formulation typically comprises one or more beta hydroxy acid(s) and/or salt(s) thereof. Non-limiting examples of beta hydroxy acids include salicylic acid, propionic acid, beta-hydroxybutyric acid, beta-hydroxy beata-methylbutyric acid, beta-phenyl-lactic acid, beta-phenylpyruvic acid, carnitine, a salt thereof, and combinations of two or more thereof. The beta hydroxy acids and/or salts thereof may in some cases be selected from salicylic acid, esters of salicylic acid, sodium salicylate, beta hydroxybutanoic acid, tropic acid, trethocanic acid, beta hydroxyl acids obtained from white willow bark extract and/or wintergreen leaves, salt(s) thereof, and combinations of two or more thereof. In some embodiments, the beta hydroxy acid(s) comprises salicylic acid and/or sodium salicylate, beta hydroxyl acids of willow bark extract, or combinations thereof. In one embodiment, the beta hydroxy acid and/or salt thereof is salicylic acid and/or sodium salicylate.

The personal care formulations of the invention may comprise one or more alpha hydroxy acid(s) and/or salt(s) thereof. The total amount of alpha hydroxy acid(s) and/or salt(s) thereof present in the personal care composition may be about 5 to about 25 wt. %, based on the total weight of the personal care composition. In some embodiments, the amount of alpha hydroxy acid(s) and/or salt(s) thereof present in the personal care composition is from about 5 to about 22 wt. %, about 5 to about 20 wt. %, about 5 to about 19 wt. %, about 5 to about 17 wt. %, about 5 to about 15 wt. %, about 5 to about 13 wt. %, about 5 to about 11 wt. %, about 5 to about 9 wt. %; from about 8 to about 25 wt. %, about 8 to about 22 wt. %, about 8 to about 19 wt. %, about 8 to about 17 wt. %, about 8 to about 15 wt. %, about 8 to about 13 wt. %, about 8 to about 11 wt. %; from about 10 to about 25 wt. %, about 10 to about 22 wt. %, about 10 to about 19 wt. %, about 10 to about 17 wt. %, about 10 to about 15 wt. %, about 10 to about 13 wt. %; from about 12 to about 25 wt. %, about 12 to about 22 wt. %, about 12 to about 19 wt. %, about 12 to about 17 wt. %, about 12 to about 15 wt. %; from about 14 to about 25 wt. %, about 14 to about 22 wt. %, about 14 to about 19 wt. %, about 14 to about 17 wt. %; from about 16 to about 25 wt. %, about 16 to about 22 wt. %, about 16 to about 19 wt. %; from about 18 to about 25 wt. %, about 18 to about 22 wt. %, or any range or subrange thereof, based on the total weight of the personal care composition.

The personal care composition may include one or more alpha hydroxy acid(s) and/or salt(s) thereof. For instance, the personal care formulation may include one or more alpha hydroxy acid(s) and/or salt(s) thereof, such as those selected from C₃ to C₇ alpha-hydroxy acid or C₄ to C₆ alpha-hydroxy acid. Examples of alpha hydroxy acid and/or salts thereof include glycolic acid, citric acid, lactic acid, malic acid, mandelic acid, ascorbic acid, alpha-hydroxybutyric acid, alpha-hydroxyisobutyric acid, alpha-hydroxyisocaproic acid, atrrolactic acid, alpha-hydroxyisovaleric acid, ethyl pyruvate, galacturonic acid, glucopehtonic acid, glucopheptono 1,4-lactone, gluconic acid, gluconolactone, glucuronic acid, glucurronolactone, glycolic acid, isopropyl pyruvate, methyl pyruvate, mucic acid, pyruvia acid, saccharic acid, saccaric acid 1,4-lactone, tartaric acid, tartronic acid, and salts thereof. In some embodiments, the personal care formulation includes a salt of an alpha-hydroxy acid. The salt of the alpha-hydroxy acid is preferably a sodium salt or a potassium salt. In at least one embodiment, the salt is a sodium salt (i.e., the cation associated with the salt is a sodium).

In certain embodiments, the one or more alpha hydroxy acid includes, but is not limited to, mandelic acid, glycolic acid, citric acid, lactic acid, malic acid, tartaric acid, phytic acid, hydroxycaprylic acid, hydroxycapric acid, glutaric acid, gluconic acid, a salt thereof, or a combination of two or more thereof. For example, the alpha hydroxy acid(s) and/or salt(s) thereof may be selected from citric acid, mandelic acid, glycolic acid, lactic acid, a salt thereof, and a combination of two or more thereof. In certain instance, the alpha-hydroxy acid is selected from mandelic acid, lactic acid, malic acid, tartaric acid, phytic acid, tartaric acid, alpha-hydroxy glutaric acid, gluconic acid, a salt thereof, and a combination of two or more thereof. Yet in further instances, the alpha-hydroxy acid or salt thereof is selected from lactic acid, malic acid, sodium-D-gluconate, a salt thereof, and a combination of two or more thereof. In at least one embodiment, the alpha hydroxy acid is lactic acid.

The personal care compositions may be formulated to have a weight ratio of the total amount of beta hydroxy acid(s) and/or salt(s) thereof to the total amount of the alpha hydroxy acid(s) and/or salt(s) thereof of about 1:1 to about 1:11; about 1:1 to about 1:10, about 1:1 to about 1:9, about 1:1 to about 1:8, about 1:1 to about 1:7, about 1:1 to about 1:6, about 1:1 to about 1:5, about 1:1 to about 1:4, about 1:1 to about 1:3, about 1:1 to about 1:2, or about 1:1.

The personal care compositions may include one or more anti-acne agent(s). The amount of anti-acne agent(s) present in the oral care composition may be from about 0.01 to about 20 wt. %, based on the total weight of the personal care composition. For instance, the personal care composition may include one or more anti-acne agent(s) in a total amount from about 0.01 to about 35 wt. %, about 0.01 to about 30 wt. %, about 0.05 to about 30 wt. %, about 0.05 to about 29 wt. %, about 0.1 to about 30 wt. %, about 0.2 to about 30 wt. %, about 0.25 to about 30 wt. %, about 0.5 to about 30 wt. %; from about 1 to about 30 wt. %, about 2.5 to about 30 wt. %, about 5 to about 30 wt. %, about 7.5 to about 30 wt. %, about 10 to about 30 wt. %, or about 12.5 to about 30 wt. %.

The amount of anti-acne agent(s) present in the oral care composition may be from about 0.01 to about 7 wt. %, based on the total weight of the personal care composition. For instance, the personal care composition may include one or more anti-acne agent(s) in a total amount from about 0.01 to about 5 wt. %, about 0.01 to about 3 wt. %, about 0.01 to about 2 wt. %, about 0.01 to about 1 wt. %, about 0.01 to about 0.75 wt. %, about 0.01 to about 0.5 wt. %, about 0.01 to about 0.25 wt. %, about 0.01 to about 0.1 wt. %; from about 0.05 to about 7 wt. %, about 0.05 to about 5 wt. %, about 0.05 to about 3 wt. %, about 0.05 to about 2 wt. %, about 0.05 to about 1 wt. %, about 0.05 to about 0.75 wt. %, about 0.05 to about 0.5 wt. %, about 0.05 to about 0.25 wt. %, about 0.05 to about 0.1 wt. %; from about 0.1 to about 7 wt. %, about 0.1 to about 5 wt. %, about 0.1 to about 3 wt. %, about 0.1 to about 2 wt. %, about 0.1 to about 1 wt. %, about 0.1 to about 0.75 wt. %, about 0.1 to about 0.5 wt. %, about 0.1 to about 0.25 wt. %; from about 0.15 to about 7 wt. %, about 0.15 to about 5 wt. %, about 0.15 to about 3 wt. %, about 0.15 to about 2 wt. %, about 0.15 to about 1 wt. %, about 0.15 to about 0.75 wt. %, about 0.15 to about 0.5 wt. %, about 0.15 to about 0.25 wt. %; from about 0.4 to about 7 wt. %, about 0.4 to about 5 wt. %, about 0.4 to about 3 wt. %, about 0.4 to about 2 wt. %, about 0.4 to about 1 wt. %, about 0.4 to about 0.75 wt. %; from about 0.8 to about 7 wt. %, about 0.8 to about 5 wt. %, about 0.8 to about 3 wt. %, about 0.8 to about 2 wt. %, about 0.8 to about 1 wt. %; from about 1 to about 2 wt. %; from about 1.2 to about 7 wt. %, about 1.2 to about 5 wt. %, about 1.2 to about 3 wt. %, about 1.2 to about 2 wt. %; from about 1.6 to about 7 wt. %, about 1.6 to about 5 wt. %, about 1.6 to about 3 wt. %, about 1.6 to about 2 wt. %; from about 2 to about 7 wt. %, about 2 to about 5 wt. %, about 2 to about 3 wt. %, or any range or subrange thereof, based on the total weight of the personal care composition.

Examples of anti-acne agents include, but are not limited to, keratolytics, such as salicylic acid, and derivatives of salicylic acid; retinoids, such as retinoic acid, tretinoin, oleyl adapalate, adapalene, tazarotene; sulfur-containing D- and L-amino acids and their derivatives and salts; lipoic acid; antibiotics and antimicrobials, such as benzoyl peroxide, triclosan, chlorhexidine gluconate, octopirox, tetracycline, 2,4,4′-trichloro-2′-hydroxy diphenyl ether, 3,4,4′-trichlorobanilide, nicotinamide, tea tree oil, rofecoxib, azelaic acid and its derivatives, phenoxyethanol, phenoxypropanol, phenoxisopropanol, ethyl acetate, clindamycin, erythromycin, and meclocycline; sebostats, such as flavonoids; and bile salts, such as scymnol sulfate and its derivatives, deoxycholate, and cholate; and combinations of two or more thereof. Examples of retinoids include tretinoin, isotretinoin, motretinide, oleyl adapalate, adapalene, tazarotene, azelaic acid, and retinol. In some embodiments, the retinoid is present in an amount of from about 0.1 wt. % to about 1 wt. %, based on the total weight of the personal care composition.

In certain embodiments, the one or more anti-acne agent comprise keratolytics, retinoids, tretinoin, adapalene, tazarotene, lipoic acid, benzoyl peroxide, triclosan, chlorhexidine gluconate, octopirox, tetracycline, 2,4,4′-trichloro-2′-hydroxy diphenyl ether, 3,4,4′-trichlorobanilide, nicotinamide, tea tree oil, rofecoxib, azelaic acid and its derivatives, phenoxyethanol, phenoxypropanol, phenoxisopropanol, ethyl acetate, clindamycin, erythromycin, meclocycline, sebostats, Minocycline, Moxifloxacin, N-acetylcystein, Nadifloxacin, octopirox, Prulifloxacin, Sitafloxacin, sodium sulfacetamide, spirinolactone, sulfacetamide, sulfur, tazarotene, tretinoin, triclosan, ulifloxacin, metronidazole, ornidazole, urea, and combinations of two or more thereof.

The personal care compositions may include two or more anti-acne agents. For instance, the personal care composition may include three, four, five, six, or seven anti-acne agents, and/or may comprise a range of anti-acne agents formed therefrom.

In some embodiments, the anti-acne agents may be are selected from acetretin, adapalene, alitretinoin, azelaic acid, Azithromycin, benzoyl peroxide, Besifloxacin, bexarotene, Cefotaxime, Cefoxitin, Ceftaroline, Ceftobiprole, Ceftriaxone, Cephalothin, clindamycin, crythromycin, etretinate, Garenoxacin, glycolic acid, isotretinoin, lactic acid, Minocycline, Moxifloxacin, N-acetylcystein, Nadifloxacin, octopirox, phenoxyethanol, phenoxypropanol, Prulifloxacin, pyruvic acid, Radezolid (RX-1741), Retapamulin, retinoic acid, Roxithromycin, salicylic acid, Sitafloxacin, sodium sulfacetamide, spirinolactone, sulfacetamide, sulfur, tazarotene, tretinoin, triclosan, ulifloxacin, metronidazole, ornidazole, urea, and a combination of two or more thereof.

The anti-acne agent may be selected from antimicrobial peptides having activity against P. acnes. The antimicrobial peptide can be a naturally occurring peptide or an analog thereof, or it can be a synthetic peptide. As used herein an “analog” refers to a naturally-occurring antimicrobial peptide that has been chemically modified to improve its effectiveness and/or reduce its toxic side effects. The antimicrobial peptide can be a peptide known to be effective against Gram positive bacteria. Non-limiting examples include lantibiotics, such as nisin, subtilin, epidermin and gallidermin; defensins; attacins, such as sarcotoxin; cecropins, such as cecropin A, bactericidin, and lepidopteran; magainins; melittins; histatins; brevinins; and combinations thereof.

In some embodiments, the anti-acne agent can be a free fatty acid (FFA), fatty acid derivatives, or fatty acid esters of propylene glycol (PG) or glycerol (G) derivatives and any combinations thereof. Examples include lauric acid, stearic acid, myristic acid, oleic acid, linoleic acid, myristoleic acid, palmitooleic acid, linoleic acid, linolenic acid, sapienic acid, different polyunsaturated FAs (PUFA), propylene glycol monolaurate, glycerol mono and/or di laurate, propylene glycol monolocate, glycerol mono and/or di oleate, and combinations thereof.

One or more antioxidants may be included in the personal care composition. For example, the personal care composition may include antioxidants in an amount from about 0.01 to about 7 wt. %, based on the total weight of the personal care composition. In some instances, the personal care composition includes the one or more anti-acne agent(s) in a total amount from about 0.01 to about 5 wt. %, about 0.01 to about 3 wt. %, about 0.01 to about 2 wt. %, about 0.01 to about 1 wt. %, about 0.01 to about 0.75 wt. %, about 0.01 to about 0.5 wt. %, about 0.01 to about 0.25 wt. %, about 0.01 to about 0.1 wt. %; from about 0.05 to about 7 wt. %, about 0.05 to about 5 wt. %, about 0.05 to about 3 wt. %, about 0.05 to about 2 wt. %, about 0.05 to about 1 wt. %, about 0.05 to about 0.75 wt. %, about 0.05 to about 0.5 wt. %, about 0.05 to about 0.25 wt. %, about 0.05 to about 0.1 wt. %; from about 0.1 to about 7 wt. %, about 0.1 to about 5 wt. %, about 0.1 to about 3 wt. %, about 0.1 to about 2 wt. %, about 0.1 to about 1 wt. %, about 0.1 to about 0.75 wt. %, about 0.1 to about 0.5 wt. %, about 0.1 to about 0.25 wt. %; from about 0.15 to about 7 wt. %, about 0.15 to about 5 wt. %, about 0.15 to about 3 wt. %, about 0.15 to about 2 wt. %, about 0.15 to about 1 wt. %, about 0.15 to about 0.75 wt. %, about 0.15 to about 0.5 wt. %, about 0.15 to about 0.25 wt. %; from about 0.4 to about 7 wt. %, about 0.4 to about 5 wt. %, about 0.4 to about 3 wt. %, about 0.4 to about 2 wt. %, about 0.4 to about 1 wt. %, about 0.4 to about 0.75 wt. %; from about 0.8 to about 7 wt. %, about 0.8 to about 5 wt. %, about 0.8 to about 3 wt. %, about 0.8 to about 2 wt. %, about 0.8 to about 1 wt. %; from about 1.2 to about 7 wt. %, about 1.2 to about 5 wt. %, about 1.2 to about 3 wt. %, about 1.2 to about 2 wt. %; from about 1.6 to about 7 wt. %, about 1.6 to about 5 wt. %, about 1.6 to about 3 wt. %, about 1.6 to about 2 wt. %; from about 2 to about 7 wt. %, about 2 to about 5 wt. %, about 2 to about 3 wt. %, or any range or subrange thereof, based on the total weight of the personal care composition.

Additionally or alternatively, the personal care composition may comprise one or more antioxidant(s). The antioxidant may be present in the personal care composition in an amount from about 0.01 to about 7 wt. %, based on the total weight of the personal care composition. For example, the personal care composition may be formulated to have a total amount of antioxidant(s) ranging from about 0.01 to about 5 wt. %, about 0.01 to about 3 wt. %, about 0.01 to about 2 wt. %, about 0.01 to about 1 wt. %, about 0.01 to about 0.75 wt. %, about 0.01 to about 0.5 wt. %, about 0.01 to about 0.25 wt. %, about 0.01 to about 0.1 wt. %; from about 0.05 to about 7 wt. %, about 0.05 to about 5 wt. %, about 0.05 to about 3 wt. %, about 0.05 to about 2 wt. %, about 0.05 to about 1 wt. %, about 0.05 to about 0.75 wt. %, about 0.05 to about 0.5 wt. %, about 0.05 to about 0.25 wt. %, about 0.05 to about 0.1 wt. %; from about 0.1 to about 7 wt. %, about 0.1 to about 5 wt. %, about 0.1 to about 3 wt. %, about 0.1 to about 2 wt. %, about 0.1 to about 1 wt. %, about 0.1 to about 0.75 wt. %, about 0.1 to about 0.5 wt. %, about 0.1 to about 0.25 wt. %; from about 0.15 to about 7 wt. %, about 0.15 to about 5 wt. %, about 0.15 to about 3 wt. %, about 0.15 to about 2 wt. %, about 0.15 to about 1 wt. %, about 0.15 to about 0.75 wt. %, about 0.15 to about 0.5 wt. %, about 0.15 to about 0.25 wt. %; from about 0.4 to about 7 wt. %, about 0.4 to about 5 wt. %, about 0.4 to about 3 wt. %, about 0.4 to about 2 wt. %, about 0.4 to about 1 wt. %, about 0.4 to about 0.75 wt. %; from about 0.8 to about 7 wt. %, about 0.8 to about 5 wt. %, about 0.8 to about 3 wt. %, about 0.8 to about 2 wt. %, about 0.8 to about 1 wt. %; from about 1.2 to about 7 wt. %, about 1.2 to about 5 wt. %, about 1.2 to about 3 wt. %, about 1.2 to about 2 wt. %; from about 1.6 to about 7 wt. %, about 1.6 to about 5 wt. %, about 1.6 to about 3 wt. %, about 1.6 to about 2 wt. %; from about 2 to about 7 wt. %, about 2 to about 5 wt. %, about 2 to about 3 wt. %, or any range or subrange thereof, based on the total weight of the personal care composition.

The one or more antioxidant(s) may comprise amellia japonica extract, quercetin, green tea, soy, milk thistle, algae, aloe, angelica, bitter orange, coffee, goldthread, grapefruit, hoellen, honeysuckle, Job's tears, lithospermum, mulberry, peony, puerarua, rice, safflower, or a combination of two or more thereof. The personal care composition may also include antioxidants selected from beta carotene, phytosterols, and combinations of two or more thereof.

Additional antioxidants include vitamin C and derivatives thereof, including ascorbic acid, sodium ascorbate, and the fat soluble esters tetrahexyldecyl ascorbate and ascorbyl palmitate, magnesium ascorbyl phosphate, ascorbyl-glucoside, glucosamine ascorbate, ascorbyl acetate, etc. Extracts from plants containing a high amount of vitamin C, such as camu berry (Myrciaria dubia), acerola, Emblica officinalis, and bioflavonoids from rose hip and citrus may in some instances be used. Further antioxidants include Sesame (Sesamum indicum) or sesame lignin (the fibrous compounds associated with the sesame). The antioxidant may be a bioflavonoids, such as hesperidin methyl chalcone.

The antioxidants may be selected from tocopherols (e.g., d-α-tocopherol, d-β-tocopherol, d-γ-tocopherol, d-delta-tocopherol), tocotrienols (e.g., d-α-tocotrienol, d-β-tocotrienol, d-γ-tocotrienol, d-delta-tocotrienol), vitamin E (α-tocopherol acetate), and combinations thereof. These compounds may be isolated from natural sources, prepared by synthetic means, or mixtures thereof. Tocotrienol-enriched vitamin E preparations may be obtained by fractionating vitamin E preparations to remove a portion of tocopherols and recover a preparation more highly concentrated in tocotrienol. Useful tocotrienols include natural products isolated, e.g., from wheat germ oil, grain, or palm oil using high performance liquid chromatography, or isolated by alcohol extraction and/or molecular distillation from barley, brewer's grain or oats. As used herein, the term “tocotrienols” includes tocotrienol-rich-fractions obtained from these natural products as well as the pure compounds. The increased glutathione peroxidase activity protects the skin from oxidative damage.

In addition, carotenoids, particularly the xanthophyll type may be useful antioxidants. The xanthopyll type carotenoids include molecules, such as lutein, canthaxantin, cryptoxanthin, zeaxanthin and astaxanthin. Xanthophylls protect compounds, such as vitamin A, vitamin E, and other carotenoids.

The personal care composition may include one or more skin soothing agent(s), such as aloe vera extract, allantoin, licorice extract, glycyrrhizin, or combinations thereof. In some preferable embodiments, the personal care compositions includes two or more skin soothing agents. For example, the personal care composition may include two, three, four, five, six, seven, or any range thereof, of skin soothing agents. When two or more skin soothing agents are incorporated into the personal care composition, preferably at least one of the skin soothing agents is licorice extract. For example, in at least one preferable embodiment, the personal care composition includes licorice extract.

In some embodiments, the soothing agents may be selected from natural extracts comprising: Centella asiatica extract, Polygonum cuspidatum root extract, Scutellaria baicalensis root extract, Camellia sinensis leaf extract, Glycyrrhiza glabra (licorice) root extract, Chamomilla recutita (matricaria) flower extract, Rosmarinus officinalis (rosemary) leaf extract, tea (Camellia sinensis) leaf extract, Poria cocos extract, Phragmites karka extract, lidocaine, chia seed extract, Rosa centifolia flower extract, aloe vera, allantoin, D-panthenol, turmeric, avocado oil, and lichen extract, or a combination of two or more thereof.

The amount of the one or more skin soothing agent(s) in the personal care composition may be about 0.01 to about 7 wt. %, based on the total weight of the personal care composition. In some instances, the personal care composition may be formulated to have skin soothing agents(s) present in a total amount of about 0.01 to about 5 wt. %, about 0.01 to about 3 wt. %, about 0.01 to about 2 wt. %, about 0.01 to about 1 wt. %, about 0.01 to about 0.75 wt. %, about 0.01 to about 0.5 wt. %, about 0.01 to about 0.25 wt. %, about 0.01 to about 0.1 wt. %; from about 0.05 to about 7 wt. %, about 0.05 to about 5 wt. %, about 0.05 to about 3 wt. %, about 0.05 to about 2 wt. %, about 0.05 to about 1 wt. %, about 0.05 to about 0.75 wt. %, about 0.05 to about 0.5 wt. %, about 0.05 to about 0.25 wt. %, about 0.05 to about 0.1 wt. %; from about 0.1 to about 7 wt. %, about 0.1 to about 5 wt. %, about 0.1 to about 3 wt. %, about 0.1 to about 2 wt. %, about 0.1 to about 1 wt. %, about 0.1 to about 0.75 wt. %, about 0.1 to about 0.5 wt. %, about 0.1 to about 0.25 wt. %; from about 0.15 to about 7 wt. %, about 0.15 to about 5 wt. %, about 0.15 to about 3 wt. %, about 0.15 to about 2 wt. %, about 0.15 to about 1 wt. %, about 0.15 to about 0.75 wt. %, about 0.15 to about 0.5 wt. %, about 0.15 to about 0.25 wt. %; from about 0.4 to about 7 wt. %, about 0.4 to about 5 wt. %, about 0.4 to about 3 wt. %, about 0.4 to about 2 wt. %, about 0.4 to about 1 wt. %, about 0.4 to about 0.75 wt. %; from about 0.8 to about 7 wt. %, about 0.8 to about 5 wt. %, about 0.8 to about 3 wt. %, about 0.8 to about 2 wt. %, about 0.8 to about 1 wt. %; from about 1.2 to about 7 wt. %, about 1.2 to about 5 wt. %, about 1.2 to about 3 wt. %, about 1.2 to about 2 wt. %; from about 1.6 to about 7 wt. %, about 1.6 to about 5 wt. %, about 1.6 to about 3 wt. %, about 1.6 to about 2 wt. %; from about 2 to about 7 wt. %, about 2 to about 5 wt. %, about 2 to about 3 wt. %, or any range or subrange thereof, based on the total weight of the personal care composition.

The personal care composition may include one or more humectant(s) e.g., in an amount ranging from about 0.01 to about 5 wt. %, based on the total weight of the personal care composition. For example, the one or more humectant(s) may be present in a total amount from about 0.01 to about 4 wt. %, about 0.01 to about 3 wt. %, about 0.01 to about 2 wt. %, about 0.01 to about 1 wt. %, about 0.01 to about 0.75 wt. %, about 0.01 to about 0.5 wt. %, about 0.01 to about 0.25 wt. %, about 0.01 to about 0.1 wt. %; from about 0.05 to about 5 wt. %, about 0.05 to about 3 wt. %, about 0.05 to about 2 wt. %, about 0.05 to about 1 wt. %, about 0.05 to about 0.75 wt. %, about 0.05 to about 0.5 wt. %, about 0.05 to about 0.25 wt. %, about 0.05 to about 0.1 wt. %; from about 0.1 to about 5 wt. %, about 0.1 to about 3 wt. %, about 0.1 to about 2 wt. %, about 0.1 to about 1 wt. %, about 0.1 to about 0.75 wt. %, about 0.1 to about 0.5 wt. %, about 0.1 to about 0.25 wt. %; from about 0.15 to about 5 wt. %, about 0.15 to about 3 wt. %, about 0.15 to about 2 wt. %, about 0.15 to about 1 wt. %, about 0.15 to about 0.75 wt. %, about 0.15 to about 0.5 wt. %, about 0.15 to about 0.25 wt. %; from about 0.4 to about 5 wt. %, about 0.4 to about 3 wt. %, about 0.4 to about 2 wt. %, about 0.4 to about 1 wt. %, about 0.4 to about 0.75 wt. %; about 0.5 wt. %; from about 0.8 to about 5 wt. %, about 0.8 to about 3 wt. %, about 0.8 to about 2 wt. %, about 0.8 to about 1 wt. %; from about 1.2 to about 5 wt. %, about 1.2 to about 3 wt. %, about 1.2 to about 2 wt. %; from about 1.6 to about 5 wt. %, about 1.6 to about 3 wt. %, about 1.6 to about 2 wt. %; from about 2 to about 7 wt. %, about 2 to about 5 wt. %, about 2 to about 3 wt. %, or any range or subrange thereof, based on the total weight of the personal care composition.

Examples of humectants that may be incorporated into the personal care composition include, but are not limited to, polyhydric alcohols such as glycerin, saccharide isomerate butylene glycol, 1,3-propanediol, sorbitol, xylitol or low molecular weight polyethylene glycols (PEGs), polyoxyethylenes or combinations thereof. In some embodiments, the humectant agents are sorbitol (preferably, non-crystal sorbitol), butylene glycol, 1,3-propanediol, glycerin (e.g., vegetable refined glycerin), polyoxyethylene glycol or combinations thereof. In at least one embodiment, the humectants are chosen from glycerin, butylene glycol and 1,3-propanediol. Typically, the humectant is vegetable refined glycerin. Other suitable humectants include, but are not limited to, polyglutamic acid, saccharide isomerate or combinations thereof. In at least one instance, the humectant is saccharide isomerate. Saccharide isomerate can be commercially obtained from, e.g., DSM under the trademark of Pentavitin™. Additional examples of humectants include glycerol and its derivatives, urea and its derivatives, sodium pidolate, xylitol, serine, sodium lactate, ectoin and its derivatives, chitosan and its derivatives, collagen, plankton, an extract of Imperata cylindra, beta-glucan, and/or rose hip oil.

In certain embodiments, the personal care composition includes a humectant selected from sorbitol, butylene glycol, 1,3-propanediol, glycerin, polyoxyethylene glycol, polyglutamic acid, saccharide isomerate, or a combination of two or more thereof. In some preferred embodiments, the personal care composition comprises saccharide isomerate.

The personal care compositions may include a carrier, such as water and/or a monoalcohol. For example, the personal care composition may include a volatile monoalcohol. In certain embodiments, the carrier may comprise ethanol, propanol, butanol, or a combination thereof. In at least one embodiment, the carrier comprises waters. In another embodiment, the carrier is ethanol. In yet a further embodiment, the carrier comprises ethanol and water.

The amount of carrier in the personal care composition may range, in some embodiments, from about 0.01 to about 7 wt. %, based on the total weight of the personal care composition. In some instances, the total amount of carrier(s) present in the personal care composition may be about 0.01 to about 5 wt. %, about 0.01 to about 3 wt. %, about 0.01 to about 2 wt. %, about 0.01 to about 1 wt. %, about 0.01 to about 0.75 wt. %, about 0.01 to about 0.5 wt. %, about 0.01 to about 0.25 wt. %, about 0.01 to about 0.1 wt. %; from about 0.05 to about 7 wt. %, about 0.05 to about 5 wt. %, about 0.05 to about 3 wt. %, about 0.05 to about 2 wt. %, about 0.05 to about 1 wt. %, about 0.05 to about 0.75 wt. %, about 0.05 to about 0.5 wt. %, about 0.05 to about 0.25 wt. %, about 0.05 to about 0.1 wt. %; from about 0.1 to about 7 wt. %, about 0.1 to about 5 wt. %, about 0.1 to about 3 wt. %, about 0.1 to about 2 wt. %, about 0.1 to about 1 wt. %, about 0.1 to about 0.75 wt. %, about 0.1 to about 0.5 wt. %, about 0.1 to about 0.25 wt. %; from about 0.15 to about 7 wt. %, about 0.15 to about 5 wt. %, about 0.15 to about 3 wt. %, about 0.15 to about 2 wt. %, about 0.15 to about 1 wt. %, about 0.15 to about 0.75 wt. %, about 0.15 to about 0.5 wt. %, about 0.15 to about 0.25 wt. %; from about 0.4 to about 7 wt. %, about 0.4 to about 5 wt. %, about 0.4 to about 3 wt. %, about 0.4 to about 2 wt. %, about 0.4 to about 1 wt. %, about 0.4 to about 0.75 wt. %; from about 0.8 to about 7 wt. %, about 0.8 to about 5 wt. %, about 0.8 to about 3 wt. %, about 0.8 to about 2 wt. %, about 0.8 to about 1 wt. %; from about 1.2 to about 7 wt. %, about 1.2 to about 5 wt. %, about 1.2 to about 3 wt. %, about 1.2 to about 2 wt. %; from about 1.6 to about 7 wt. %, about 1.6 to about 5 wt. %, about 1.6 to about 3 wt. %, about 1.6 to about 2 wt. %; from about 2 to about 7 wt. %, about 2 to about 5 wt. %, about 2 to about 3 wt. %, or any range or subrange thereof, based on the total weight of the personal care composition.

Some embodiments of the present invention provide personal care compositions that are substantially free, or free, of resorcinol. Further embodiments of the present invention provide personal care composition that are substantially free, or free, of hydroquinone. Yet other embodiments provide personal care compositions that are substantially free, or free, of resorcinol and hydroquinone.

In some embodiments, the personal care composition comprises a meroterpene, e.g., bakuchiol. In some embodiments, the bakuchiol is present in an amount of from about 0.1 wt. % to about 5 wt. %, based on the total weight of the personal care composition. In some embodiments, the bakuchiol is present in an amount of from about 0.2 wt. % to about 4 wt. %, based on the total weight of the personal care composition. In some embodiments, the bakuchiol is present in an amount of from about 0.3 wt. % to about 3.5 wt. %, based on the total weight of the personal care composition. In some embodiments, the bakuchiol is present in an amount of from about 0.4 wt. % to about 3 wt. %, based on the total weight of the personal care composition. In some embodiments, the bakuchiol is present in an amount of from about 0.5 wt. % to about 2.5 wt. %, based on the total weight of the personal care composition. In some embodiments, the bakuchiol is present in an amount of from about 1 wt. % to about 2 wt. %, based on the total weight of the personal care composition. The bakuchiol may, in some instances, be in the form of a salt.

In some embodiments, the personal care compositions are in a form having the one or more carrier(s) in an amount ranging from about 25 to about 85 wt. %, based on the total weight of the personal care composition. For example, the one or more carrier(s) may be present in the personal care composition in an amount from about 30 to about 85 wt. %, about 35 to about 85 wt. %, about 40 to about 85 wt. %, about 45 to about 85 wt. %, about 50 to about 85 wt. %, about 55 to about 85 wt. %, about 60 to about 85 wt. %, about 65 to about 85 wt. %, about 70 to about 85 wt. %; from about 25 to about 80 wt. %, about 30 to about 80 wt. %, about 35 to about 80 wt. %, about 40 to about 80 wt. %, about 45 to about 80 wt. %, about 50 to about 80 wt. %, about 55 to about 80 wt. %, about 60 to about 80 wt. %, about 65 to about 80 wt. %; from about 25 to about 75 wt. %, about 30 to about 75 wt. %, about 35 to about 75 wt. %, about 40 to about 75 wt. %, about 45 to about 75 wt. %, about 50 to about 75 wt. %, about 55 to about 75 wt. %, about 60 to about 75 wt. %; from about 25 to about 70 wt. %, about 30 to about 70 wt. %, about 35 to about 70 wt. %, about 40 to about 70 wt. %, about 45 to about 70 wt. %, about 50 to about 70 wt. %, about 55 to about 70 wt. %; from about 25 to about 65 wt. %, about 30 to about 65 wt. %, about 35 to about 65 wt. %, about 40 to about 65 wt. %, about 45 to about 65 wt. %, about 50 to about 65 wt. %; from about 25 to about 60 wt. %, about 30 to about 60 wt. %, about 35 to about 60 wt. %, about 40 to about 60 wt. %, about 45 to about 60 wt. %; from about 25 to about 55 wt. %, about 30 to about 55 wt. %, about 35 to about 55 wt. %, about 40 to about 55 wt. %; from about 25 to about 50 wt. %, about 30 to about 50 wt. %, about 35 to about 50 wt. %, or any range or subrange thereof, based on the total weight of the personal care composition.

In some embodiments, polyhydric alcohols and polyether derivatives may be used as solvents and/or surfactants in the topical formulations. Suitable polyhydric alcohols and polyethers include propylene glycol, di propylene glycol, polypropylene glycols 2000 and 4000, poly(oxyethylene-co-oxypropylene) glycols, glycerol, sorbitol, ethoxylated sorbitol, hydroxypropylsorbitol, polyethylene glycols 200-6000, methoxy polyethylene glycols 350, 550, 750, 2000 and 5000, polyethylene oxide] homopolymers (100,000-5,000,000), polyalkylene glycols and derivatives, hexylene glycol, 2-methyl-2,4-pentanediol, 1,3-butylene glycol, 1,2,6-hexanetriol, 2-ethyl-1 ,3-hexanediol, vicinal glycols having 15 to 18 carbon atoms, and polyoxypropylene derivatives of trimethylolpropane.

In some embodiments, solvents increase penetration possibly by enclosing the polar pathway and/or by fluidizing lipids. Examples include water alcohols (e.g., methanol and ethanol); alkyl methyl sulfoxides (e.g., dimethyl sulfoxide, alkyl homo logs of methyl sulfoxide dimethyl acetamide and dimethyl formamide); pyrrolidones (e.g., 2 pyrrolidone, N-methyl, 2-purrolidone); laurocapram (Azone), miscellaneous solvents (e.g., propylene glycol, glycerol, silicone fluids, isopropyl palmitate).

In some embodiments, surfactants may enhance polar pathway transport, especially of hydrophilic drugs. The ability of a surfactant to alter penetration is a function of the polar head group and the hydrocarbon chain length. Anionic surfactants include, but are not limited to, Dioctyl sulphosuccinate, Sodium lauryl sulphate, and Decodecylmethyl sulphoxide. Nonionic surfactants include, but are not limited to, Pluronic F127, and Pluronic F68. Bile salts include, but are not limited to, Sodium ms taurocholate, Sodium deoxycholate, and Sodium tauroglycocholate. Others include, for example, Propylene glycol-oleic acid and 1,4-butane diol-linoleic acid, urea, N,N-dimethyl-m-toluamide, calcium thioglycolate; anticholinergic agents, cucalyptol, di-o-methyl-P-cyclodextrin and soyabean casein.

In other embodiments, polyhydric alcohol esters may be used as emulsifiers or emollients. Suitable polyhydric alcohol esters include ethylene glycol mono- and di-fatty acid esters, diethylene glycol mono- and di-fatty acid esters, polyethylene glycol (200-6000) mono- and di-fatty acid esters, propylene glycol mono- and di-fatty esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol di stearate, polyoxyethylene polyol fatty acid ester, sorbitan fatty acid esters, and polyoxyethylene sorbitan fatty acid esters.

Suitable emulsifiers for use in topical formulations include anionic, cationic, nonionic, and zwitterionic surfactants. Preferred ionic emulsifiers include phospholipids, such as lecithin and derivatives. Lecithin and other phospholipids may be used to prepare liposomes containing the peptide compositions as described herein. Formation of lipid vesicles occurs when phospholipids such as lecithin are placed in water and consequently form one bilayer or a series of bilayers, each separated by water molecules, once enough energy is supplied. Liposomes can be created by sonicating phospholipids in water. Low shear rates create multilamellar liposomes. Continued high-shear sonication tends to form smaller unilamellar liposomes. Hydrophobic chemicals can be dissolved into the phospholipid bilayer membrane. The lipid bilayers of the liposomes deliver the peptide compositions as described herein.

The personal care compositions described herein may contain micelles, or an aggregate of surfactant molecules dispersed in an aqueous solution. Micelles may be prepared by dispersing an oil solvent in an aqueous solution comprising a surfactant, where the surfactant concentration exceeds the critical micelle concentration. The resulting formulation contains micelles, i.e., spherical oil droplets surrounded by a membrane of polar surfactant molecules, dispersed in the aqueous solvent.

Sterols including, for example, cholesterol and cholesterol fatty acid esters; amides such as fatty acid amides, ethoxylated fatty acid amides, and fatty acid alkanolamides may also be used as emollients and/or penetration enhancers.

In some embodiments, the personal care compositions described herein have a pH of from about 2.0 to about 4.5. In other embodiments, the personal care compositions described herein have a pH of from about 2.1 to about 4.4. In further embodiments, the personal care compositions described herein have a pH of from about 2.2 to about 4.3. In certain embodiments, the personal care compositions described herein have a pH of from about 2.3 to about 4.2. In some embodiments, the personal care compositions described herein have a pH of from about 2.4 to about 4.1. In other embodiments, the personal care compositions described herein have a pH of from about 2.5 to about 4.0. In certain embodiments, the personal care compositions described herein have a pH of from about 2.6 to about 3.9. In further embodiments, the personal care compositions described herein have a pH of from about 2.7 to about 3.8. In other embodiments, the personal care compositions described herein have a pH of from about 2.8 to about 3.7. In some embodiments, the personal care compositions described herein have a pH of from about 2.9 to about 3.6. In certain embodiments, the personal care compositions described herein have a pH of from about 3.0 to about 3.5. In other embodiments, the personal care compositions described herein have a pH of from about 2.1 to about 4.1. In further embodiments, the personal care compositions described herein have a pH of from about 3.1 to about 4.1. In some embodiments, the personal care composition has a pH of about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, or about 4.5.

### EXAMPLES

### Example 1

A non-limiting example personal care composition (Ex. 1) was prepared in accordance with aspect of the invention. The formulation of Ex. 1 is shown in Table 1 (below).

TABLE 1

| | Ex. 1 |
|---|---|
| Ingredient | (wt. %) |
| Lactic Acid | 5-20 |
| Salicylic Acid | 5-20 |
| Resorcinol | 0.1-20 |
| Licorice Extract | 0.01-0.2 |
| Alpha Lipoic Acid | 0.01-0.2 |
| Oleyl Adapalate | 0.1-0.4 |
| Saccharide Isomerate | 0.01-0.1 |
| Carrier (water and/or ethanol) | Q.S. to 100 |

### Example 2

A second non-limiting example personal care composition (Ex. 2) was prepared in accordance with aspect of the invention. The formulation of Ex. 2 is shown in Table 1 (below).

TABLE 2

| | Ex. 2 |
|---|---|
| Ingredient | (wt. %) |
| Lactic Acid | 5-20 |
| Mandelic Acid | 5-20 |
| Salicylic Acid | 0.1-5 |
| Lactobionic Acid | 0.5-7.5 |
| Licorice Extract | 0.01-1 |
| Alpha Lipoic Acid | 0.01-1 |
| Oleyl Adapalate | 0.05-1 |
| Saccharide Isomerate | 0.1-1 |
| Bakuchiol | 0.1-5 |
| Carrier (water and/or ethanol) | Q.S. to 100 |

### Example 3

In vitro evaluations of Ex. 1 were conducted to assess the effect of such skin care composition on expression of TGFB2, VEGF, and AQP3, which are biomarkers associated with the regeneration and improved healing. Specifically, Ex. 1 was applied to samples of keratinocytes provided by EpiDerm™M and incubated for 24 or either 48 hours. Control samples were developed by preparing keratinocyte samples according to the same procedure but without applying Ex. 1.

Additionally, Ex. 1 was applied to skin explant samples obtained from a 37 year old female volunteer having a Fitzpatrick skin type of V. The skin explant samples were then incubated for 6 days. Control samples were developed by preparing skin explant samples according to the same procedure but without applying Ex. 1.

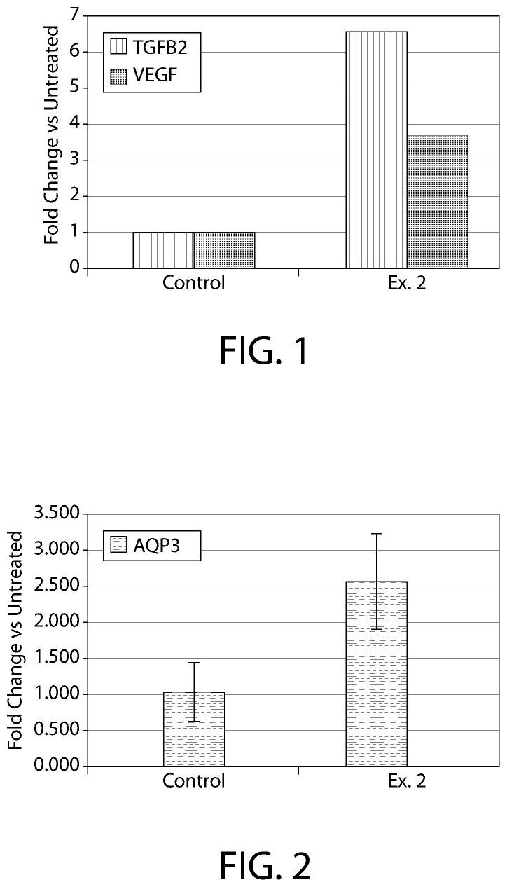

The keratinocyte samples and skin explant samples were evaluated and a summary of the results are shown in FIGS. 1 and 2. Based on these results, it was determined that Ex. 1 promotes desired modulation of biomarkers involved in skin cell regeneration and wound healing, which are compromised in acne lesions.

### Example 4

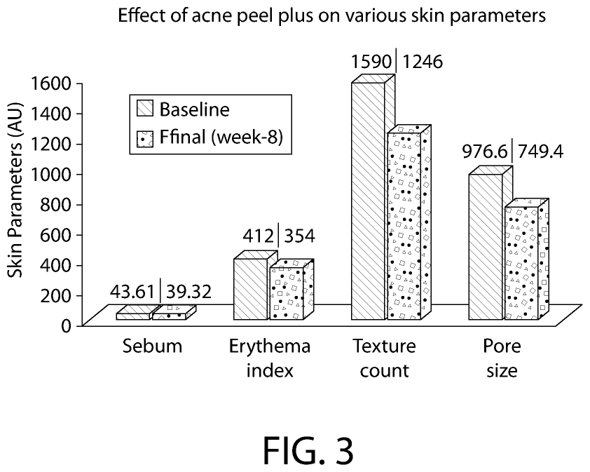

In vivo evaluations of Ex. 1 were conducted to assess the effect on such skin care composition over an eight-week period. Specifically, five volunteers were instructed to apply Ex. 1 to an area of skin after rinsing and pat drying the area of skin twice. The volunteers were then instructed to apply a provided moisturizer and a provided sunscreen on the same area of skin. The area of skin was evaluated to assess the amount of sebum, erythema index, texture count, and pore size. A summary of the amount of sebum, erythema index, texture count, and pore size before and upon completion of the eight-week period of applying Ex. 1 is shown in FIG. 3. Based on the results, it was determined that Ex. 1 is effective for reducing acne lesions, showing sebum, pores, uneven texture, and redness.

The present disclosure has been described with reference to exemplary embodiments. Although a limited number of embodiments have been shown and described, it will be appreciated by those skilled in the art that changes may be made in these embodiments without departing from the principles and spirit of the preceding detailed description. It is intended that the present disclosure be construed as including all such modifications and alterations insofar as they come within the scope of the appended claims or the equivalents thereof.

1-33. (canceled)

34. A personal care composition comprising:
oleyl adapalate;
a beta hydroxy acid and/or a salt thereof; and
a humectant.

35. The personal care composition according to claim 34, wherein the beta hydroxy acid and/or a salt thereof comprises salicylic acid, beta hydroxybutanoic acid, tropic acid, trethocanic acid, a salt thereof, or a combination of two or more thereof.

36. The personal care composition according to claim 34, wherein the beta hydroxy acid and/or a salt thereof comprises salicylic acid, a salt thereof, or a combination of thereof.

37. The personal care composition according to claim 34, wherein the beta hydroxy acid and/or a salt thereof is present in amount from about 5 to about 25 wt. %, based on the total weight of the personal care composition.

38. The personal care composition according to claim 34 further comprising an alpha hydroxy acid and/or salt thereof.

39. The personal care composition according to claim 38, wherein the alpha hydroxy acid and/or salt thereof comprises mandelic acid, lactic acid, malic acid, tartaric acid, phytic acid, a salt thereof, or a combination of two or more thereof.

40. The personal care composition according to claim 38, wherein the alpha hydroxy acid and/or salt thereof is present in an amount ranging from about 5 to about 25 wt. %, based on the total weight of the personal care composition.

41. The personal care composition according to claim 34, further comprising an anti-acne agent selected from keratolytics, retinoids, tretinoin, adapalene, tazarotene, lipoic acid, benzoyl peroxide, triclosan, chlorhexidine gluconate, octopirox, tetracycline, 2,4,4′-trichloro-2′-hydroxy diphenyl ether, 3,4,4′-trichlorobanilide, nicotinamide, tea tree oil, rofecoxib, azelaic acid and its derivatives, phenoxyethanol, phenoxypropanol, phenoxisopropanol, ethyl acetate, clindamycin, erythromycin, meclocycline, sebostats, Minocycline, Moxifloxacin, N-acetylcystein, Nadifloxacin, octopirox, Prulifloxacin, Sitafloxacin, sodium sulfacetamide, spirinolactone, sulfacetamide, sulfur, tazarotene, tretinoin, triclosan, ulifloxacin, metronidazole, ornidazole, urea, and a combination of two or more thereof.

42. The personal care composition according to claim 34 further comprising an antioxidant selected from amellia japonica extract, quercetin, green tea, soy, milk thistle, algae, aloe, angelica, bitter orange, coffee, goldthread, grapefruit, hoellen, honeysuckle, Job's tears, lithospermum, mulberry, peony, puerarua, rice, safflower, and a combination of two or more thereof.

43. The personal care composition according to claim 34, wherein the humectant is selected from sorbitol, butylene glycol, 1,3-propanediol, glycerin, polyoxyethylene glycol, polyglutamic acid, saccharide isomerate, and a combination of two or more thereof.

44. The personal care composition according to claim 43, wherein the humectant is present in an amount from about 0.01 to about 5 wt. %, based on the total weight of the personal care composition.

45. The personal care composition according to claim 34 further comprising licorice extract.

46. A personal care composition comprising:
oleyl adapalate;
an alpha hydroxy acid and/or salt thereof;
lipoic acid and/or a salt thereof; and
saccharide isomerate.

47. The personal care composition according to claim 46, wherein the alpha hydroxy acid and/or salt thereof is selected from mandelic acid, lactic acid, malic acid, tartaric acid, phytic acid, a salt thereof, and a combination of two or more thereof.

48. The personal care composition according to claim 46 further comprising a beta hydroxy acid and/or salt thereof selected from salicylic acid, beta hydroxybutanoic acid, tropic acid, trethocanic acid, a salt thereof, and a combination of two or more thereof.

49. The personal care composition according to claim 46, further comprising licorice extract.

50. A personal care composition comprising:
a retinoid;
bakuchiol;
an alpha hydroxy acid, or a salt thereof; and
a cosmetically acceptable carrier.

51. The personal care composition according to claim 50, wherein the alpha hydroxy acid, or salt thereof, is selected from mandelic acid, lactic acid, malic acid, tartaric acid, phytic acid, a salt thereof, and a combination of two or more thereof.

52. The personal care composition according to claim 50, wherein the retinoid comprises oleyl adapalate, or a derivative thereof.

53. The personal care composition according to claim 50. having a pH of from about 2.5 to about 4.5.
